(19) United States
(12) Patent Application Publication
(10) Pub. No.: US 2004/0028637 A1
(43) Pub. Date: Feb. 12, 2004

(54) Use as thickeners in cosmetics of neutralized copolymers comprising weak acid units and strong acid units, and cosmetic compositions comprising same

(76) Inventors: Emmanuel Villard, Saint-Etienne (FR); Frederic Blondel, Saint-Etienne (FR)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/311,996

(22) PCT Filed: Jun. 15, 2001
(86) PCT No.: PCT/FR01/01892

(30) Foreign Application Priority Data
Jun. 23, 2000 (FR) 00/08201

(51) Int. Cl. A61K 7/06; A61K 7/11

(52) U.S. Cl. 424/70.16

(57) ABSTRACT

This invention relates to a cosmetic, dermatologic, pharmaceutical, or veterinary composition comprising at least one thickening and/or emulsifying polymer for an aqueous medium, such composition being obtained by polymerization: at least one unsaturated weak acid and at least one unsaturated strong acid such as AMPS, in the presence of a cross-linking or branching agent and optionally of a transfer agent and characterized by

a water-soluble polymer fraction ranging from 5 to 50 percent by weight, preferably 8 to 35 percent.

These acids are partially neutralized at the time of polymerization.

The composition obtained surprisingly exhibits optimal cosmetic characteristics, in particular very high stability in the presence of electrolytes over a very wide pH range (1-13) as a result of selection of suitable polymerization conditions for the thickening and/or emulsifying polymer (neutralization rate, active matter concentration, strong/weak acid molar ratio).

This invention relates to the technical sector of cosmetic, dermatological, pharmaceutical or veterinary, and detergent compounds comprising at least one thickening and/or emulsifying polymer for aqueous media.

The following have been disclosed in the prior art:

use of cross-linked polymers for increasing the viscosity of cosmetic, pharmaceutical, or technical products (FR 2,416,723),

use of a polymer dispersion as paste thickener for textile imprinting (EP 0,161,038),

compositions comprising a thickening polymer based on non-ionic monomers such as acrylic acid or cationic monomers swelling in water. The polymers are obtained in the form of a reversal emulsion. Applications such as cosmetics and above all textile or velvet pile imprinting are cited, among other applications (GB 2,077,750),

thickening polymers obtained in the form of a reversal emulsion on the basis of strong acids (U.S. Pat. No. 4,172,066),

EP 0 503 853, which relates to a thickener based on AMPS and acrylamide,

use of a monomer having a strong acid specifically for thickening acid media (petroleum application) (GB 2,110,744),

the process of preparing a compound in the form of inverse latex from an anionic polyelectrolyte (homopolymer), branched or cross-linked, based on a monomer performing a strong acid function (WO 99/42521).

The information in EP 0 186 361 has also been published.

An extremely important technical problem in the industry in question is that of imparting to the final composition certain properties of interest such as the feel and the stability of the viscosity in the presence of electrolytes. Despite the existence of a strong demand, the prior art has yet to contribute a solution to this problem.

Hence the corresponding technical problem is that of compounds possessing the capability of thickening aqueous compounds which may be used in cosmetics, dermatology, in the veterinary or pharmaceutical area, and in similar areas of body care products, and in particular ones which improve the feel and stability.

Specifically, there is a demand for both acid pH and basic pH thickening compounds, especially ones efficient at very acid pH values, in particular a pH value of approximately 1 to 3, this corresponding to a particular technical problem which is not properly solved by products of the prior art, despite significant demand by the industry.

Throughout this application “thickening and/or emulsifying polymer” is to be understood to mean a copolymer obtained by polymerization in the form of a reversal emulsion such as the one described in patent EP 0,161,038.

However, the stability of the compound in the presence of electrolytes leaves room for improvement. In effect the viscosity (and so the consistency) of the finished product tends to drop sharply upon coming into contact with electrolytes, the skin, for example. This situation results in disadvantages of comfort of use and performance.

A problem which is even more acute and of extreme importance in the industry in question is thus that of imparting to the final composition certain important properties such as stability toward electrolytes. Despite the existence of a strong demand, the prior art has failed to provide adequate satisfaction regarding the problem. Thus, when characteristics termed those of being “very cool to the touch” are described, they are in fact the direct result of a bad behavior of the viscosity of the polymer in the presence of electrolytes.

The invention relates to a family of polymer thickeners and/or emulsifiers such as those specified in EP 0 161 038 referred to above, which permit thickening of cosmetic, dermatologic, pharmaceutical, or veterinary compounds at both acid and basic pH values while imparting to the composition optimal cosmetic characteristics, ones never obtained, such as the feel and the stability of viscosity in the presence of electrolytes. Use of polymers of this type may also be considered for thickening any type of aqueous medium, in a laundering operation, for example.

The present invention relates to use of a specific selection of this family of copolymers as agents thickening and/or emulsifying (in whole or in part) cosmetic, dermatologic, and pharmaceutical compounds, for human or veterinary use, or detergents, as well as cosmetic, dermatologic, and pharmaceutical compounds for human or veterinary use, or detergents, containing at least one of these polymers as thickener(s) and/or emulsifier(s).

The compositions claimed for the invention contribute significant additional improvement in this specific area and include as thickener and/or emulsifier at least one polymer of this family of thickeners and/or emulsifiers obtained by polymerization of

5 to 95 molar percent of at least one monomer performing a weak acid function

and 5 to 95 molar percent of at least one monomer performing a strong acid function,

and characterized in that they have a fraction of water-soluble polymers ranging from 5 to 50 per by weight (of the total polymer), preferably 8 to 35 percent.

The composition obtained surprisingly exhibits optimal cosmetic characteristics, including in particular a very high stability in the presence of electrolytes over a very wide range of pH values (1-13°), as a result of suitable selection of thickener and/or emulsifier polymerization conditions (neutralization rate, percentage of soluble polymers, strong acid/weak acid molar ratio, concentration of active matter) resulting in the soluble polymer fraction in question.

Hence the invention relates to use as thickener and/or emulsifier (in whole or in part) of cosmetic, dermatologic, and pharmaceutical compositions, for human or veterinary use, or as detergents, of at least one polymer obtained by polymerization of

5 to 95 molar percent of at least one monomer performing a weak acid function, and

5 to 95 molar percent of at least one monomer performing a strong acid function and

characterized in that it exhibits a fraction of water-soluble polymers ranging from 5 to 50 percent by weight (in relation to the total polymer), preferably from 8 to 35 percent.

While not wishing to advance any theory, the Applicant does believe that this characteristic, which is extremely important at the industrial and commercial levels, is to be attributed to the high proportion of water-soluble polymers.

The most closely related commercial products, which advertise “a cool touch,” and so moderate resistance of the electrolytes of the skin, have a content of water-soluble polymers not exceeding 2 to 3 percent. There does then appear to be a threshold of such properties of around 5 percent (cfr the 6 percent obtained in a test conducted in accordance with the invention). (The tests conducted in accordance with the invention yield values which are the mean values obtained from three tests, in order to reduce the margin of error by the greatest amount possible.)

It is also to be noted that the polymers claimed in this application have no neutral monomers; this is probably a factor in obtaining the advantages referred to in the foregoing.

In one absolutely preferred embodiment the polymers are also obtained in the presence of a cross-linking or branching agent and optionally in that of a transfer agent.

In one preferred embodiment the polymerization is carried out in a water-in-oil reversal emulsion.

In one preferred embodiment the cross-linking rate (or branching rate, as applicable) is 50 to 3,000 parts per million (one the basis of methylene bisacrylamide or MBA) relative to the polymer or an equivalent cross-linking with a cross-linking agent of different efficiency, in accordance with parameters with which the expert is familiar.

Under the same conditions the polymer which would be obtained in the absence of the cross-linking agent would have an intrinsic viscosity (I.V.) of 3 to 25 dl/g (deciliters per gram).

In another embodiment the polymerization concentration is 15 to 55 percent by weight.

In yet another embodiment the polymerization is conducted optionally in the presence of a transfer agent.

The expert will be able to determine on the basis of his own knowledge the extent to which the transfer of cross-linking or branching agent is to be used to obtain a branched or cross-linked polymer. The expert also knows the chemical definition of these two categories of polymers; many earlier papers and patents have dealt with such polymers. In this application “cross-linked or branched” or “cross-linked” and similar words will be used to refer to the foregoing.

In addition, it is essential that at the time of polymerization neutralization of all monomers performing an acid function be partial and range from 5 to 95 percent. The pH of polymerization varies with the neutralization rate. Hence it is claimed for the invention that it is possible to work at a pH at which the problems of corrosion of industrial equipment are very sharply reduced, to the extent that special equipment is not required. The process will be conducted in particular at pH>4 in polymerization, preferably 4.2 to 4.5; it is to be noted that at pH values below 4 (even though the difference in pH in terms of absolute value may appear to be very small), special anticorrosive equipment is not necessary. Hence the option (non-restrictive) provided by the invention of working at polymerization pH values slightly higher than 4 represents another interesting and surprising technical threshold.

Thanks to appropriate choice of conditions for polymerization of the thickener and/or emulsifier (neutralization rate, concentration of active substance, strong/weak molar acid ratio), the composition obtained surprisingly exhibits optimal cosmetic characteristics such as feel and stability over a very wide range of pH values (1-13) while retaining a very strong and efficient thickening capability, even at a very acid pH value, in particular around pH 1 to 3.

The expert will understand optimization of the polymerization conditions by reading this description and because of his individual knowledge, or as a result of simple routine tests.

In addition, it is also possible to concentrate or to isolate the polymer by all known techniques.

In particular, there are many processes for obtaining powder on the basis of soluble polymer emulsions or ones which swell in water, these processes consisting of isolating the active matter from other constituents of the emulsion. Processes such as the following may be cited; this list is non-restrictive:

precipitation in a non-solvent medium such as acetone, methanol, and other polar solvents. Simple filtration then permits isolation of the polymer particle.

azeotropic distillation in the presence of an agglomerating agent and stabilizing polymer which makes it possible to obtain agglomerates which are easily isolated by filtration before drying of the particle is undertaken.

“Spray drying,” or drying by atomization or pulverization, which consists of creating a cloud of fine droplets of emulsion in a stream of hot air for a controlled period.

In a preferred embodiment the copolymer is obtained on the basis of:

10 to 80 molar percent of at least one monomer performing a weak acid function.

and 20 to 90 molar percent of a monomer performing a strong acid function.

In one particular embodiment the neutralization rate (at the time of polymerization) of the aggregate of unsaturated acids is preferably 10 to 80 percent.

By way of preeminent preference a cross-linking or branching agent and optionally a transfer agent are incorporated.

In another particular embodiment the cross-linking rate preferably ranges from 100 to 800 ppm (on the basis of MBA) relative to the polymer or equivalent cross-linking with a cross-linking agent of different efficiency.

In the absence of a cross-linking agent, the intrinsic IV viscosity of the polymer obtained is preferably 4 to 20 dl/g.

The polymerization concentration is preferably 25 to 45 percent active matter by weight.

The overall rate of neutralization of monomers performing an acid function ranges from 20 to 90 percent on polymerization.

At the time of polymerization in the form of water-in-oil emulsion, the continuous phase used may be an oil or a solvent of mineral origin and/or synthesized and/or of plant origin. Use is to be made preferably of a solvent or a non-mineral oil.

Following is a non-restrictive list of strong acid monomers:

monomers performing a sulfonic acid or phosphonic acid functions, such as 2-acrylamido-2-methyl propane sulfonic acid (AMPS).

Following is a non-restrictive list of monomers performing a weak acid function: acrylic acid, methacrylic acid, itaconic acid, crotonic acid, maleic acid, fumaric acid, etc.

Following is a non-restrictive list of cross-linking agents: methylene bisacrylamide (MBA), ethylene glycol diacrylate, polyethylene glycol dimethacrylate, diacrylamide, cyanomethylacrylate, vinyl oxyethylacrylate or methacrylate and formaldehyde, glyoxal, compounds of the glycidyl ether type such as ethyleneglycol diglycidyl ether, or the epoxyes or any other means familiar to the expert permitting cross-linking.

Following is a nonrestrictive list of transfer agents: isopropyl alcohol, sodium hypophosphite, mercaptoethanol, etc.

It will be seen that it will be within the capability of the expert to select, under the conditions and within the areas of application indicated above, optimal conditions such that the final polymer has a water-soluble polymer fraction ranging from 5 to 50 percent by weight of the total polymer (as determined by a metering method such as that described on page 8 of patent EP 0 343 840). The expert will know in particular how to estimate, on the basis of his own knowledge, the amount of transfer agent and cross-linking or branching agent to be used in order to obtain a final polymer having an adequate fraction of water-soluble polymer and the neutralization rate to be selected in order to obtain a polymerization agent slightly higher than 4 (if desired).

The expert will be able to select the best combination on the basis of his own knowledge and this description, as well as the following examples.

### EXAMPLES OF POLYMERS

Each of the polymers described in what follows was obtained by polymerization in the form of a reversal emulsion such as that described in EP 0,161,038. To permit better comparison, the cross-linking and limiting agent rates have been kept constant.

In the light of the results obtained it appears that the behavior (thickening power and ability to resist electrolytes) is directly a function of the polymerization conditions, and in particular of the neutralization rate and of the molar ratio of strong acid to weak acid.

Choice of the polymer to be used is then a function of the characteristics required for the finished composition.

| | P1 | P2 | P3 | P4 | P5 | P6 | X |
|---|---|---|---|---|---|---|---|
| Molar % | 60 | 60 | 60 | 40 | 50 | 90 | 57 |
| AMPS | | | | | | | |
| (strong acid) | | | | | | | |
| Molar % | 40 | 40 | 40 | 60 | 50 | 10 | 43 |
| Acrylic | | | | | | | |
| Acid | | | | | | | |
| (weak acid) | | | | | | | |
| % Neutrali- | 70 | 50 | 36 | 55 | 60 | 70 | 57 |
| zation | | | | | | | |
| (total) | | | | | | | |
| % Active | 35 | 35 | 30 | 30 | 30 | 35 | 38 |
| Matter | | | | | | | |
| % Water- | 17 | 6 | 15 | 10 | 28 | 20 | 3 |
| soluble | | | | | | | |
| Polymer | | | | | | | |
| QC1 | 6000 | 16500 | 12000 | 3250 | 4500 | 12500 | 12000 |
| (viscosity | | | | | | | |
| measurement | | | | | | | |
| at pH = 3) | | | | | | | |
| QC2 (drop in | 54 | 70 | 43 | 54 | 44 | 60 | 99.9 |
| viscosity | | | | | | | |
| from addi- | | | | | | | |
| tion of salts) | | | | | | | |

It would undoubtedly be technically possible to increase the proportion of water-soluble polymers beyond 28 percent, but without great benefit, since it appears that the compromise limit is reached at around 25-30-35 percent, depending on the details of the application.

On the other hand, as already indicated, there obviously appears to be a property threshold among disclosed products (at 2 to 3 percent) and the example of the invention at 6 percent.

Additionally, it is quite surprising to find that such a clear-cut threshold exists between 3 and 6 percent, one which could neither have been foreseen nor is logical.

The group of water-soluble polymers may thus range preferably from 6 to 35 percent, and again preferably between 8 and 30-35 percent.

QC1: viscosity measurement at pH=3

Preparation of a solution A with 1 percent of active substance in deionized water, followed by adjustment of the pH value to fall between 2.9 and 3.1 by using 1N hydrochloric acid

Measurement of viscosity No. 1 by means of a Brookfield RVT viscosimeter; the values obtained are expressed in centipoises

QC2: drop in viscosity from addition of salts

A saline solution (with 70 g/l NaCl) is added to solution A in order to obtain a final salt concentration of 10 percent by mass relative to the active matter (polymer).

Measurement of viscosity No. 2 by means of a Brookfield RVT viscosimeter; the values obtained are expressed in centipoises

The drop in viscosity is calculated as follows:

Drop in viscosity (percent)=100×((viscosity 1−viscosity 2)/viscosity 1)

It is to be noted that the products of examples P1 to P6 surprisingly exhibit good stability of their viscosity in the presence of electrolytes, which is not the case with product X. The water-soluble polymer rate appears to play a role of prime importance. Because of this good resistance, the polymers claimed for this invention retain a high viscosity even at an extremely acid pH, in contrast to the known products on the market.

### EXAMPLES OF COMPOSITIONS

Within the framework of the composition examples, the two polymers used correspond to the polymer described above designated as P1. This polymer was selected because it represents a good compromise between values QC1 and QC2. In is nevertheless conceivable that one or the other of the polymers presented might be used; this selection depends exclusively on the final characteristics required for the composition.

Component A: cross-linked polymer of the P1 type comprising 60 percent AMPS and 40 percent acrylic acid used in the form of a distilled emulsion containing 62.6 percent polymer

Component B: same polymer as polymer A used in the form of a powder obtained by spray drying

The names of the ingredients used in the compositions are those given in the INCI (International Nomenclature of Cosmetic Ingredients).

### Example 1

### Gel/Creme Base, pH 3.6:

| | | percent by weight: |
|---|---|---|
| Aqueous phase: | glycerine | 2% |
| | water (aqua) | QSP 100 |
| | citric acid | QS pH = 3.5 |
| Component A | | 3% |
| Preservatives | | QS |
| Fragrance | | QS |
| Citric acid | | QS, final pH = 3.6 |

[QSP 100 = amount sufficient to make up 100 [%]]
[QS = sufficient amount]

Method of Preparation:

First stage: preparation of the aqueous phase.

Second stage: adjustment of aqueous phase pH value by addition of acid.

Third stage: component A is added during agitation.

Fourth stage: preservatives and fragrance are added.

Fifth stage: readjustment of pH to 3.6

Final Characteristics of Composition

pH=3.6

viscosity (RVT6, 20 rpm)=10,250 cps [centipoise]

appearance: white, opaque, shiny gel/cream

feel: oily, because this gel/cream breaks down very slowly on the surface of the skin.

Texture: light, not greasy, and not tacky

easily grasped by the hand, while the texture is relatively fluid

### Example 2

### Silicone-Based Lotion, pH 6

| | percent by weight: |
|---|---|
| dimethicone | 10% |
| octyl palmitate | 5% |
| water (aqua) | QSP 100 |
| Component A | 1.5% |
| Preservatives | QS |
| Fragrance | QS |

Method of Preparation:

The ingredients are incorporated in the order indicated. Component A, the preservatives, and then the fragrance are added during agitation.

Final Characteristics of Composition:

pH=5.8

viscosity (RVT 6, 20 rpm)=9,500 cps

appearance: shiny cream, easy to grasp in the hand

feel: oily and light; does not turn to “water” when applied. Final feel velvety

### Example 3

### Capillary-Based Cream, pH 5:

| | percent by weight: |
|---|---|
| water (aqua) | QSP 100 |
| olive oil (Olea Europaea) | 2% |
| Component B | 1.25% |
| Preservatives | QS |
| Fragrance | QS |
| Citric acid | QS pH = 5 |

Method of Preparation:

Component B is poured into water. Oil is then added during agitation. The preservatives and the fragrance are added when the preparation is homogenous. The pH value is set at 5.

Final Characteristics of Composition:

pH=5

viscosity (RVT 6, 20 rpm)=11,500 cps

### Example 4

### Oil/Water Makeup Removal Base, pH 4.7

| | | percent by weight: |
|---|---|---|
| Aqueous phase: | Disodium laureth sulfosuccinate | 4% (active matter) |
| | glycerin | 3% |
| | water (aqua) | QSP 100 |
| Oily phase: | caprylic/capric triglyceride | 6% |
| | soft almond oil (Prunus | 2% |
| | Amigdalus Dulcis_) | |
| Component A | | 4% |
| Preservatives | | QS |
| Fragrance | | QS |

Method of Preparation:

The aqueous phase is prepared. The oily phase is then incorporated. Component A, then the preservatives and the fragrance are added during agitation.

Final Characteristics of Composition:

pH=4.7

viscosity (RVT 6, 20 rpm)=4,000 cps

appearance: milk white

feel: foams little, easy to rinse off, easy to spread while retaining a consistent non-greasy and non-adhesive texture.

### Example 5

### Oil/Water Emulsion Base

| | | percent by weight: |
|---|---|---|
| oily phase: | “Shea butter” (Butyrospermum | 2% |
| | Parkii) (karite butter) | |
| | octyl stearate | 8% |
| | mineral oil (paraffinum liquidum) | 4% |
| water (aqua) | | QSP 100 |
| Component A | | 2% |
| Preservatives | | QS |
| Fragrance | | QS |
| Citric acid | | QS pH 4.2 |

Method of Preparation:

Preparation of oily phase at 50° C. Water is added and the temperature is held at 50° C. Component A is added during agitation.

The mixture is then brought to ambient temperature in order to add the preservatives and the fragrance. The pH value is then set.

Final Characteristics of Composition:

pH=4.2

viscosity (RVT 6, 20 rpm)=2,750 cps

appearance: opaque white fluid

feel: light and consistent; permits rubbing without formation of particles causing discomfort to consumer

### Example 6

### Gel/Cream Base, pH 10

| | percent by weight: |
|---|---|
| water (aqua) | QSP 100 |
| Component A | 3% |
| NaOH | QS pH = 10 |
| Preservatives | QS |
| Fragrance | QS |

Method of Preparation:

Component A is poured into water. The pH value is set at 10. The preservatives and the fragrance and then added during agitation.

Final Characteristics of Composition:

pH=10

viscosity (RVT 6, 20 rpm)=12,000 centipoise

appearance: white, opaque, and shiny gel/cream

feel: oily. The gel is easy to spread.

The examples presented in the foregoing demonstrate the variety of the compositions which may be targeted for use of this type of polymers, because of both their thickening and/or their emulsifying properties.

A large number of improvements are made:

The thickening and/or emulsifying polymers such as those defined may be incorporated at any temperature. In addition, they provide a greater amount of flexibility as regards the stage of incorporation.

They are efficient thickening and emulsifying polymers and thus are excellent stabilizing substances for compositions containing silicone, vegetable oils, ingredients in saline form or containing salts, or ingredients with a pH value smaller than 6.

Each of the final compositions tested exhibits optimal cosmetic characteristics such as feel and stability.

The feels resulting from the different formulations are oily: at the time of application, the texture of the composition claimed for the invention “breaks down” more slowly, this making it possible to avoid too “watery” a feel (one which resembles water), which holds little attraction for the consumer, especially in terms of efficiency and comfort of the composition.

This property also makes it possible to prevent the composition applied to the surface of the skin, hair, nails, hair, or body hairs from flowing out too quickly. Hence it is easier to control application and spreading of the composition on the surface to be treated.

It is to be noted that there is a vast selection of packaging for each of the possible formulas: pump bottle, tube, spray, jar, etc.

In addition, use of a polymer in powder form is an additional choice. It permits the benefit of the properties of the polymer such as described above while preventing the presence of the oily phase (polymer solvent) in the final composition. This possibility provides additional variety making it possible to meet new technical and/or marketing demands.

1. Use as thickener and/or emulsifier (in whole or in part) of cosmetic, dermatologic, pharmaceutical composition for human or veterinarian use, or as detergents, of at least one polymer obtained by polymerization of:
5 to 95 molar percent of at least one monomer performing a weak acid function, and of
5 to 95 molar percent of at least one monomer performing a strong acid function and
characterized in that such composition exhibits a fraction of water-soluble polymers ranging from 5 to 50 percent by weight (of the total polymer), preferably from 6 to 35 percent, preferably from 8 to 30-35 percent.

2. Use of a polymer as specified in claim 1, wherein the polymerization is performed in a water-in-oil reversal emulsion.

3. Use of a polymer as specified in claim 1 or 2, wherein the polymers are obtained also in the presence of a cross-linking or branching agent and optionally in that of a transfer agent.

4. Use of a polymer as specified in any of claims 1 to 3, wherein the cross-linking or branching rate is 50 to 3,000 parts per million (in the case of methylene bisacrylamide or MBA) relative to the polymer or an equivalent cross-linking with a cross-linking agent of different efficiency.

5. Use of a polymer as specified in any of claims 1 to 4, wherein the polymerization concentration is 15 to 55 percent by weight.

6. Use of a polymer as specified in any of claims 1 to 5, wherein the polymerization is optionally conducted in the presence of a transfer agent.

7. Use of a polymer as specified in any of claims 1 to 6, wherein, at the time of polymerization, neutralization of the totality of the monomers performing an acid function is partial and ranges from 5 to 95 percent.

8. Use of a polymer as specified in any of claims 1 to 7, wherein such polymer is in isolated or concentrated form, in particular as a result of obtaining powder on the basis of polymer emulsions which are soluble or swell in water, by isolating the active ingredient of the other constituents of the emulsion.

9. Use of a polymer as specified in claim 8, wherein use is made of one of the following processes for recovery of such polymer:
precipitation in a non-solvent medium such as acetone, methanol, and other polar solvents; simple filtration then permits isolation of the polymer particle.
azeotropic distillation in the presence of a binding agent and a stabilizing polymer which results in production of conglomerates which are isolated by filtration before drying of the particle is carried out.
spray-drying or drying by atomization or pulverization consisting of creation of a cloud of fine droplets of emulsion in a flow of hot air over a controlled period.

10. Use of a polymer as specified in any of claims 1 to 9, wherein such polymer is obtained on the basis of:
10 to 80 molar percent of at least one monomer performing a weak acid function and
20 to 90 molar percent of at least one monomer performing a
strong acid function
and optionally also in the presence of a cross-linking or branching agent and optionally of a transfer agent.

11. Use of a polymer as specified in any of claims 1 to 10, wherein the rate of neutralization (at the time of polymerization) of the entirety of the unsaturated acids of such polymer is preferably 10 to 80 percent.

12. Use of a polymer as specified in any of claims 1 to 11, wherein the rate of cross-linking or branching of such polymer ranges preferably from 100 to 800 parts per million (for MBA) relative to the polymer or an equivalent cross-linking with a cross-linking agent of different efficiency.

13. Use of a polymer as specified in any of claims 1 to 12, wherein, under the same conditions, the polymer which would be obtained in the absence of the cross-linking agent would have an intrinsic viscosity (I.V.) of 3 to 25 deciliters per gram.

14. Use of a polymer as specified in any of claims 1 to 13, wherein the polymerization concentration is preferably 25 to 45 percent active matter by weight.

15. Use of a polymer as specified in any of claims 1 to 14, wherein the total rate of neutralization of monomers performing an acid function ranges from 20 to 90 percent at the time of polymerization.

16. Use of a polymer as specified in any of claims 1 to 15, wherein, at the time of polymerization in the form of a water-in-oil emulsion, the continuous phase used may be an oil or a solvent of mineral origin and/or synthesized and/or of plant origin, preferably a non-mineral solvent or oil.

17. Use of a polymer as specified in any of claims 1 to 16, wherein such polymer includes as monomer one of the following strong-acid monomers:
monomers performing a function of the sulfonic acid type or phosponic acid type, such as 2-acrylamido-2-methylpropane sulfonic acid (AMPS).

18. Use of a polymer as specified in any of claims 1 to 17, wherein such polymer includes as monomer one of the following monomers of the weak acid type:
acrylic acid, methacrylic acid, itaconic acid, crotonic acid, maleic acid, fumaric acid.

19. Use of a polymer as specified in any of claims 1 18, wherein such polymer has been cross-linked (or branched) by one of the following cross-ling agents: methylene bisacrylamide (MBA), ethylene glycol diacrylate, polyethylene glycol dimethacrylate, diacrylamide, cyanomethacrylate, vinyloxyethacrylate or methacrylate, and formaldehyde, glyoxal, compositions of the glycidylether type such as ethyleneglycol diglycidylether, or epoxyes.

20. Use of a polymer as specified in any of claims 1 to 19, wherein such polymer includes one of the following transfer agents: isopropyl alcohol, sodium hypophosphite, mercaptoethanol.

20. Use of a polymer as specified in any of claims 1 to 19, wherein such polymer includes one of the following transfer agents: isopropyl alcohol, sodium hypophosphite, mercaptoethanol.

21. Use of a polymer as specified in any of claim 1 to 20, wherein such polymer is formed as a reversal emulsion on the basis of:

| | P1 | P2 | P3 | P4 | P5 | P6 |
|---|---|---|---|---|---|---|
| Molar % AMPS | 60 | 60 | 60 | 40 | 50 | 90 |
| (strong acid) | | | | | | |
| Molar % Acrylic | 40 | 40 | 40 | 60 | 50 | 10 |
| Acid | | | | | | |
| (weak acid) | | | | | | |
| % Neutralization | 70 | 50 | 36 | 55 | 60 | 70 |
| (total) | | | | | | |
| % Active Matter | 35 | 35 | 30 | 30 | 30 | 35 |
| % Water-soluble | 17 | 6 | 15 | 10 | 28 | 20 |
| Polymer | | | | | | |
| QC1 (viscosity | 6000 | 16500 | 12000 | 3250 | 4500 | 12500 |
| measurement at | | | | | | |
| pH = 3) | | | | | | |
| QC2 (drop in | 54 | 70 | 43 | 54 | 44 | 60 |
| viscosity from | | | | | | |
| addition of salts) | | | | | | |

22. Cosmetic, pharmaceutical, dermatologic, compositions, for human or veterinary use, or detergents, of high stability even in the presence of electrolytes, characterized in that such compositions contain as thickener(s) and/or emulsifier(s) at least one of the polymers described in any of claims 1 to 21.
